(19) United States
(12) Patent Application Publication
(10) Pub. No.: US 2006/0089708 A1
(43) Pub. Date: Apr. 27, 2006

(54) Venous bi-valve

(76) Inventors: Francisco Jose Osse, Sao Paulo (BR); Patricia E Thorpe, Iowa City, IA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/509,826

(22) PCT Filed: Feb. 20, 2003
(86) PCT No.: PCT/US03/05107

§ 371 Date: Aug. 31, 2005

Related U.S. Application Data
(60) Provisional application No. 60/359,021, filed on Feb. 20, 2002.

(51) Int. Cl. A61F 2/06

(52) U.S. Cl. 623/1.24

(57) ABSTRACT

A replacement venous valve assembly having over-the-wire or other deployable configurations of struts and membranes.

### FIELD OF THE INVENTION

The present invention relates to venous valve replacement and, in particular, to replacement venous valves to lower extremities and a therapeutic method of treating venous circulatory disorders.

### BACKGROUND OF THE INVENTION

Chronic venous insufficiency (CVI) of the lower extremities is a common condition that is considered a serious public health and socioeconomic problem. In the United States, approximately two million workdays are lost each year, and over 2 million new cases of venous. thrombosis are recorded each year. About 800,000 new cases of venous insufficiency syndrome will also be recorded annually. Ambulatory care costs of about $2,000, per patient, per month, contribute to the estimated U.S. cost of $16,000,000 per month for the treatment of venous stasis ulcers related to CVI.

It is estimated that greater than 3% of the Medicare population is afflicted by a degree of CVI manifested as non-healing ulcers. Studies have indicated that about 40% of seriously affected individuals cannot work or even leave the house except to obtain medical care: It is estimated that 0.2% of the American work force is afflicted with CVI.

Chronic venous insufficiency arises from long duration venous hypertension caused by valvular insufficiency and/or venous obstruction secondary to venous thrombosis. Other primary causes of CVI include varicosities of long duration, venous hypoplasia and arteriovenous fistula. The signs and symptoms of CVI have been used to classify the degree of severity of the disease, and reporting standards have been published. Studies demonstrate that deterioration of venous hemodynamic status correlates with disease severity. Venous reflux, measured by ultrasound studies, is the method of choice of initial evaluation of patients with pain and/or swelling in the lower extremities. In most serious cases of CVI, venous stasis ulcers are indicative of incompetent venous valves in all systems, including superficial, common, deep and communicating veins. This global involvement affects at least 30% of all cases. Standard principles of treatment are directed at elimination of venous reflux. Based on this observation, therapeutic intervention is best determined by evaluating the extent of valvular incompetence, and the anatomical distribution of reflux. Valvular incompetence, a major component of venous hypertension, is present in about 60% of patients with a clinical diagnosis of CVI.

Endovascular valve replacement refers to a new concept and new technology in the treatment of valvular reflux. The concept involves percutaneous insertion of the prosthetic device under fluoroscopic guidance. The device can be advanced to the desired intravascular location using guide wires and catheters. Deployment at a selected site can be accomplished to correct valvular incompetence. Percutaneous placement of a new valve apparatus provides a less invasive solution compared to surgical transposition or open repair of a valve.

The modern concept of a stent was introduced in the 1960s. Subsequently, it has been successfully incorporated in the treatment of arterioral aneurysms and occlusive disease. The use of endovascular stents represents one of the most significant changes in the field of vascular surgery since the introduction of surgical graft techniques in the early 1950s.

Initially, the dominant interest of vascular specialists was application of stents in the arterial system. The venous system and venous disease were not considered an arena for stent application. The utilization of endovascular treatment in venous disease was initially confined to the treatment of obstruction, in the pelvic veins (for CVI) as well as treatment of obstructed hemodialysis access grafts and decompression of portal hypertension (TIPS). Although these procedures enjoy widespread application, the actual number of patients involved is relatively low compared to the number afflicted with CVI and related syndrome. Thus, the necessity for therapy using endovascular technology for the treatment of venous disease arose. The prevalence of CVI and the magnitude of its impact demand development of an effective alternative therapy. Other examples are detailed in a published PCT application, WO 02/087467 which is incorporated by reference.

### BRIEF DESCRIPTION OF THE DRAWINGS

FIG. 1 is a photograph of one embodiment of the invention model from a side view.

FIG. 2 is a photograph of one embodiment of the invention model from a top view.

FIG. 3 is a photograph of one embodiment of the invention model from a bottom view.

FIG. 4 is a photograph of one embodiment of the invention model from a front view.

FIG. 5 is a photograph of one embodiment of the invention model from a side view.

FIG. 6 is a drawing of the closed invention positioned on delivery device.

FIG. 7 is a drawing of one embodiment of the invention without membranes.

FIG. 8 is a drawing of the partially opened invention.

FIG. 9 is a drawing of the fully opened invention positioned on a delivery device.

FIG. 10 is a drawing of a bell shaped embodiment.

FIG. 11 is top view of FIG. 10.

FIG. 12 is an fully open view of the bell shaped embodiment on a delivery device.

FIG. 13A is the bulbous embodiment of the invention positioned at valve site

FIG. 13B is an embodiment of the invention within a vein.

FIG. 14 is the fully opened bulbous embodiment on a deliver device.

FIG. 15 shows the detail of the struts without the membranes.

FIG. 16 is a top view of FIG. 15.

FIG. 17 is the bulbous embodiment with secondary struts positioned at a valve site.

FIG. 18 is the bulbous embodiment with secondary struts fully opened.

FIG. 19 is the bulbous embodiment without the membranes.

FIG. 20 is a top view of FIG. 19.

FIG. 21 is a cross section of a drawn venous valve.

FIGS. 22A and 22B are embodiments with struts and support wings positioned inside a vein.

FIG. 23 is a top view of FIG. 22B with secondary struts.

FIG. 24 is a side view of FIG. 22B.

FIG. 25 is an embodiment of FIGS. 7, 8 and 9 with secondary struts showing progression from closed to fully open modes.

### SUMMARY OF THE INVENTION

A venous valve assembly is formed with curved support struts and valve material.

### DETAILED DESCRIPTION OF THE INVENTION

Within the field of endovascular treatment, no previous technology has effectively used a replacement valve which also acts similar to a stent in a percutaneously located assembly. Indeed, recognition of the need for such a device, system and method of employment has been lacking. Attempts at venous valve repair are not common. Indeed, minimally invasive repair or replacement procedures are quite uncommon. This is due, in part, to the poor availability of properly sized and properly designed prosthetic venous valves. U.S. Pat. No. 5,500,014 discusses different attempts to provide prosthetic venous valves, and such discussion is incorporated by reference herein. For the anatomy of venous valves, an excellent reference includes Venous Valves, by R. Gottlub and R. May, published by Springer Verlag, Austria, 1986.

The inventors have devised a device, system and method of deployment for a valve assembly utilizing various materials having excellent cost, biocompatibility, and ease of use. In one embodiment, a stent is assembled having excellent length and stability characteristics, as well as an improved profile for ease of placement and automatic deployment at a deployment site. The assembly does not rely on placement at a previous valvular site but may also be utilized either proximate or distal to the incompetent valve site due to the self-expanding features and improved anti-migration characteristics of the assembly.

The use of the material chosen for endovascular valve replacement in this assembly represents a unique application of a biocompatible substance. Whether the material is formed of elastomer, sclera, small intestine sub-mucosa (SIS), other mammalian tissue, or other biocompatible material, the venous stent device of this invention will serve as a substitute for deteriorated venous valves which have been altered by thrombosis or congenital hypoplasia. The valve prosthesis which self-expands similar to a stent will be percutaneously introduced with a small sized catheter delivery system. Justification for development of this invention is based on the incidence of venous disorders that lack adequate endovascular therapy. Patients who are treated surgically undergo a more invasive method that involves greater costs and more numerous potential complications. The minimally invasive technique of this invention will decrease length of hospital stay, lower over-all costs and permit an almost immediate return to normal activity. Indeed, it is believed that the availability of this treatment will dramatically alter the lives of many people, including those who might not have been able to undergo previous surgical techniques for the repair or replacement of damaged venous valves.

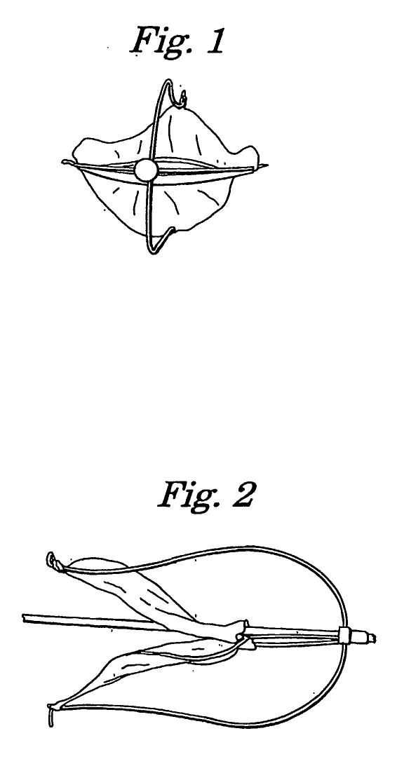

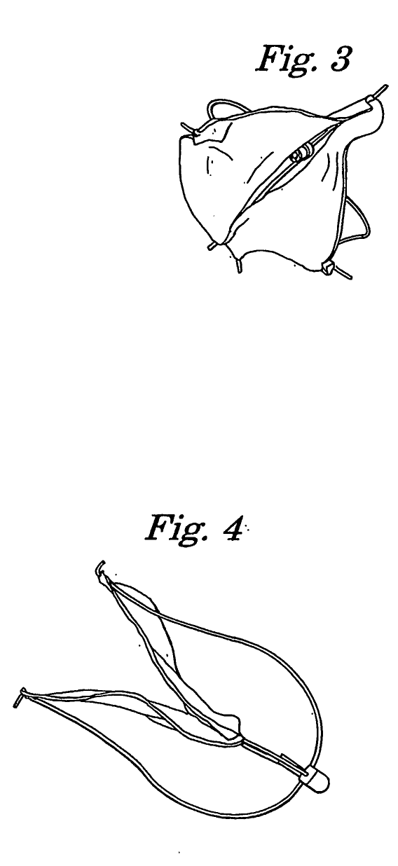

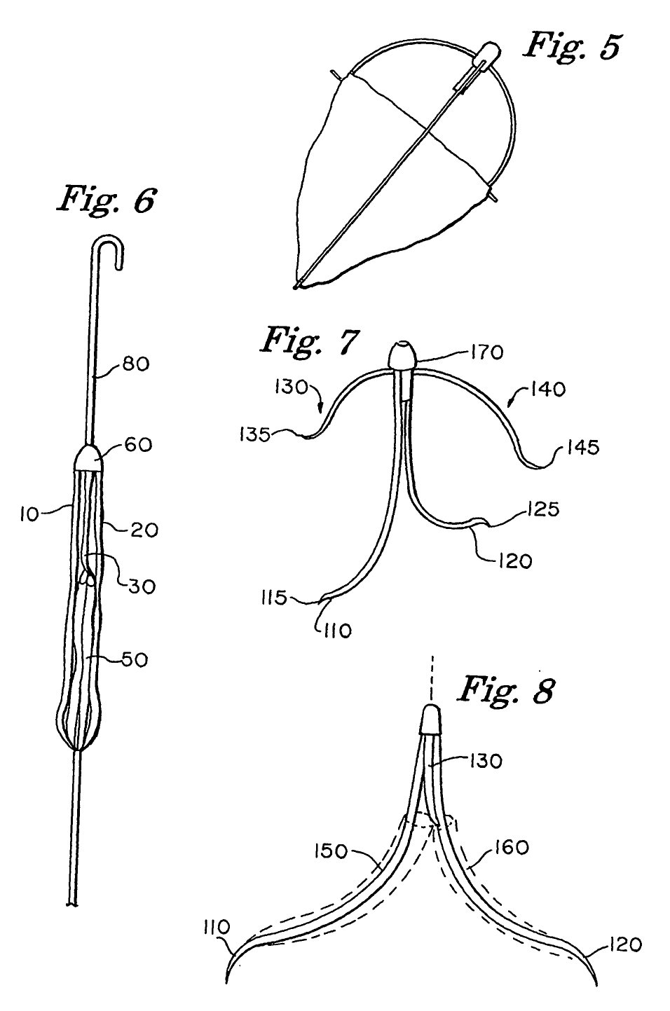

FIGS. 1-5 are photographs of one embodiment of a model of the invention taken from different angles. The features of this invention are explained in the following figures.

FIG. 6 shows a folded or otherwise contracted embodiment of the invention mounted on a guiding apparatus. The figure shows first strut 10, second strut 20, third strut 30 with the outline of the membrane 50. The connector 60 for the strut has the guiding apparatus 80 passing through the center of the connector and extending past the distal point of the struts relative to connector.

FIG. 7 shows the invention without the valve membranes. There is the first strut 110 with the membrane connection point 115, the second strut 120 with the membrane connection point 125, the third strut 130 with the connection point 135 and the fourth strut 140 with the connection point 145. This figure shows the curvature of the first and second struts curving inward towards the axis defined as going through the center of the connector 170 of the strut. This figure also shows the third and fourth struts in a plane perpendicular to the plane defined by the first and second struts.

FIG. 8 shows a partially opened venous valve assembly with the first strut 110 and the second strut 120 in an open position and showing a side view with the third strut 130 still in closed position. The first membrane 150 is shown by the dotted outline in a folded position around the first strut 110. A second membrane 160 is shown attached to the first strut and the third strut in a folded position around the second strut 120.

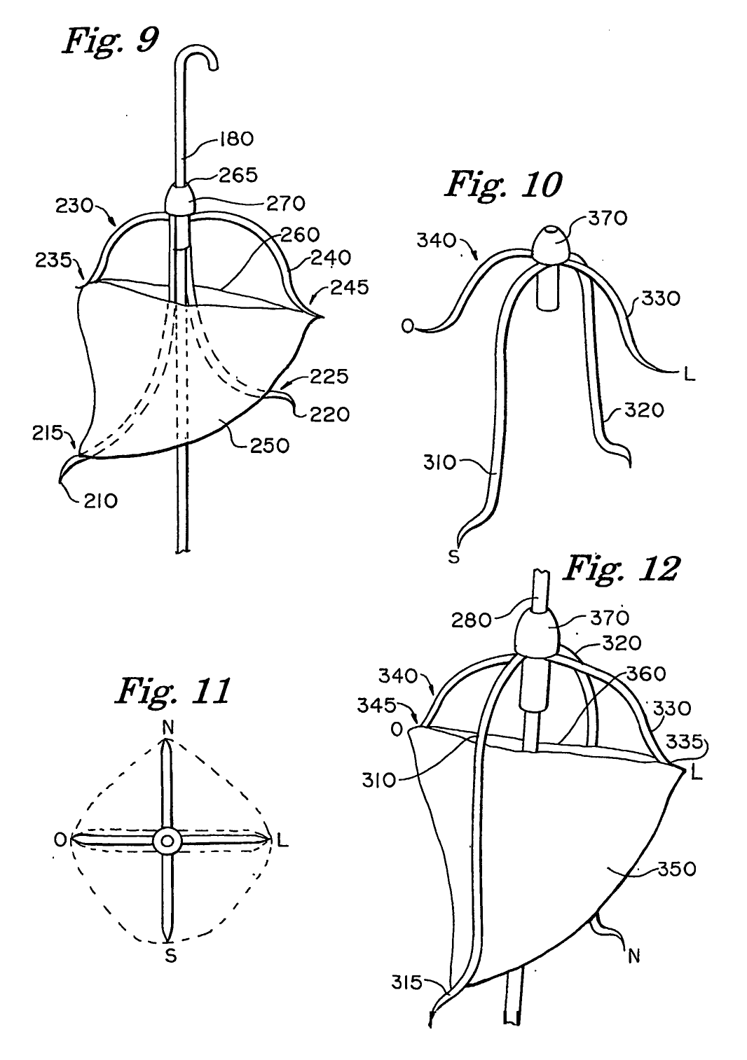

FIG. 9 is a fully opened venous valve mounted on a guiding apparatus 180. This slightly oblique view shows the membrane 250 attached to the first strut 220 at point 215 attached to the third strut 230 at point 235 attached to the fourth strut 240 at point 245. The other membrane 260 has its edge shown opposite to the membrane 250 and also shows the connection point 225 at second strut 220. Connector 270 shows the aperture 265 which the guiding device 180 is placed through in order to mount the venous valve onto the guiding device.

FIG. 10 shows another embodiment of venous valve. The first strut 310 and the generally opposite second strut 320 form a curved bell-shape in order to fit more snugly against the wall of the vein. The third strut 330 and the fourth strut 340 are shown in their perpendicular position all connected to the central connector 370.

FIG. 11 shows a top side view of FIG. 10 where the dotted lines indicate the position of the membrane on the venous valve at their respective attachment points on the struts. The top of connector 370 is facing outward from, or normal to, the page.

FIG. 12 shows the fully extended or deployed venous valve assembly with the outwardly curving first strut 310 generally opposite the other outwardly curving second strut 320; with the membrane 350 positioned between the first strut and the central access of the invention. Membrane 350 is connected to the first strut at point 315, to the third strut 330 at connection point 335, and to the fourth strut 340 at connection point 345. Also in the figure is the second strut 320, the upper boarder of the opposite membrane 360 shown in relation to the central connector 370, and the guiding apparatus 280.

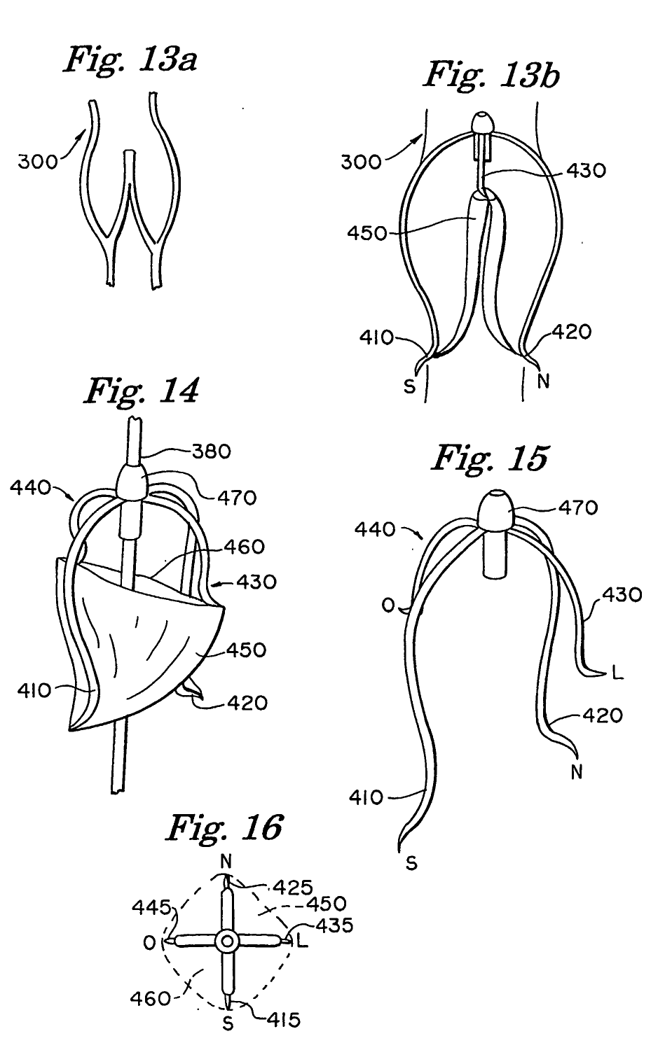

FIGS. 13A and 13B show a natural human venous valve 300 and the position of a preferred embodiment of the venous valve within the vein at the same location of the venous valve 300. The first strut 410 shows a more bulbous configuration in order to mimic the typical formation of a venous valve. The second strut 420 displays the same sort of bulbous curvature. The figure also shows the third strut 430 as a side view showing the membrane 450 in position connected to the first strut and the third strut.

FIG. 14 shows the expanded bulbous embodiment with third strut 410, second strut 420, third strut 430 and the fourth strut 440 connected to central connector 470. The membrane 450 is shown and connected to the struts similarly to FIG. 12. This figure also shows another edge of the opposite membrane 460 which is attached to the second strut 420 as well as the third strut and fourth strut.

FIG. 15 is the venous valve without the membranes attached to better show the position of first strut 410, second strut 420, third strut 430 and fourth strut 440 with the central connector 470.

FIG. 16 shows a top view of the bulbous embodiment with the dotted lines outlining the membranes 450 and 460 and showing attachment points for the first strut 415 the connection point of the third strut 435 and the connection point for membranes for the fourth strut at point 445.

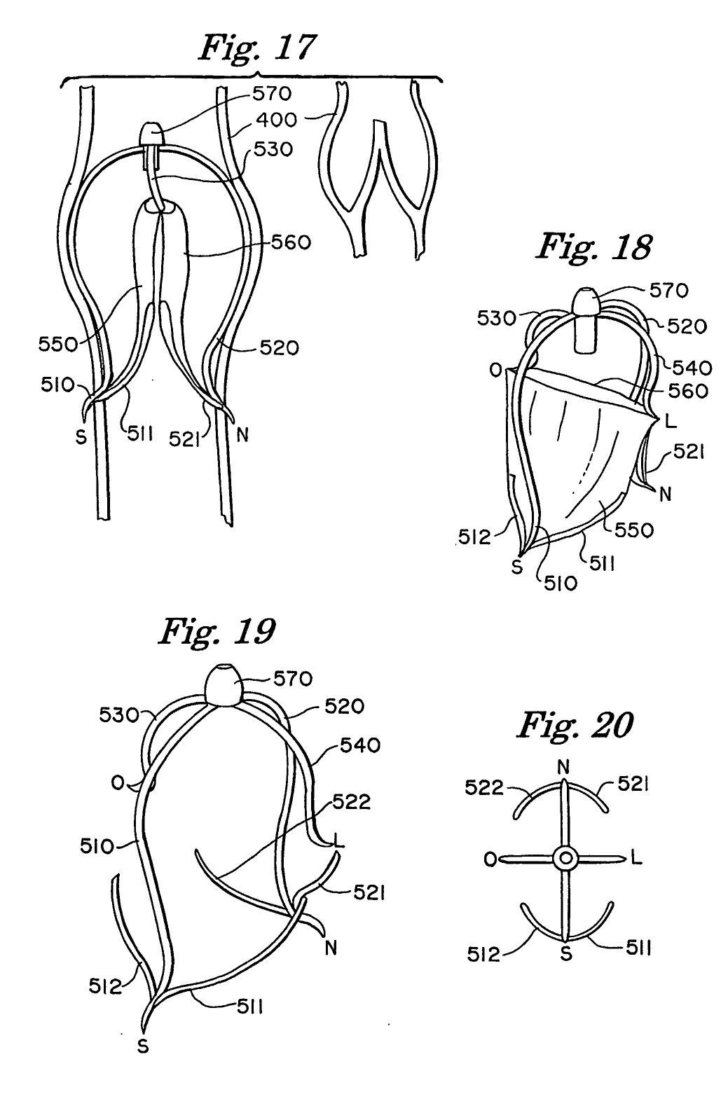

FIG. 17 shows the venous valve 400 along with another embodiment of a conformal bell-shaped venous valve placed in situ. The figure illustrates first strut 510 with a secondary strut 511, second strut 520 has also a secondary strut 521 and the position of one membrane 550 and the opposite membrane 560. In this side view is seen the third strut 530 and the central connector 570. The secondary struts act as greater contact with the vein wall to prevent unintended repositioning after desired placement.

FIG. 18 shows the bulbous embodiment with the secondary struts attached in relation to the valve membrane. First strut 510 has the first secondary strut 511 with the second secondary strut 512 with the membrane 550 attached to the first strut and third strut and the fourth strut 540. Also shown is the second strut 520 with the opposite membrane attached at point 521 in the upper edge of the second opposite membrane showing at 560.

FIG. 19 shows the bulbous venous valve without the attached membrane. What is shown is the first strut 510 with the first secondary strut 511 and the second secondary strut 512. The second strut 520 also shows the first secondary strut 521 and the second secondary strut 522 attached distally relative to the central connector 570. Also shown in relative view is the third strut 530 and the fourth strut 540.

FIG. 20 is a top view of the venous valve with secondary struts showing the first strut's, secondary struts at 511 and 512 which are shown as opposites to the second strut's secondary struts 521 and 522.

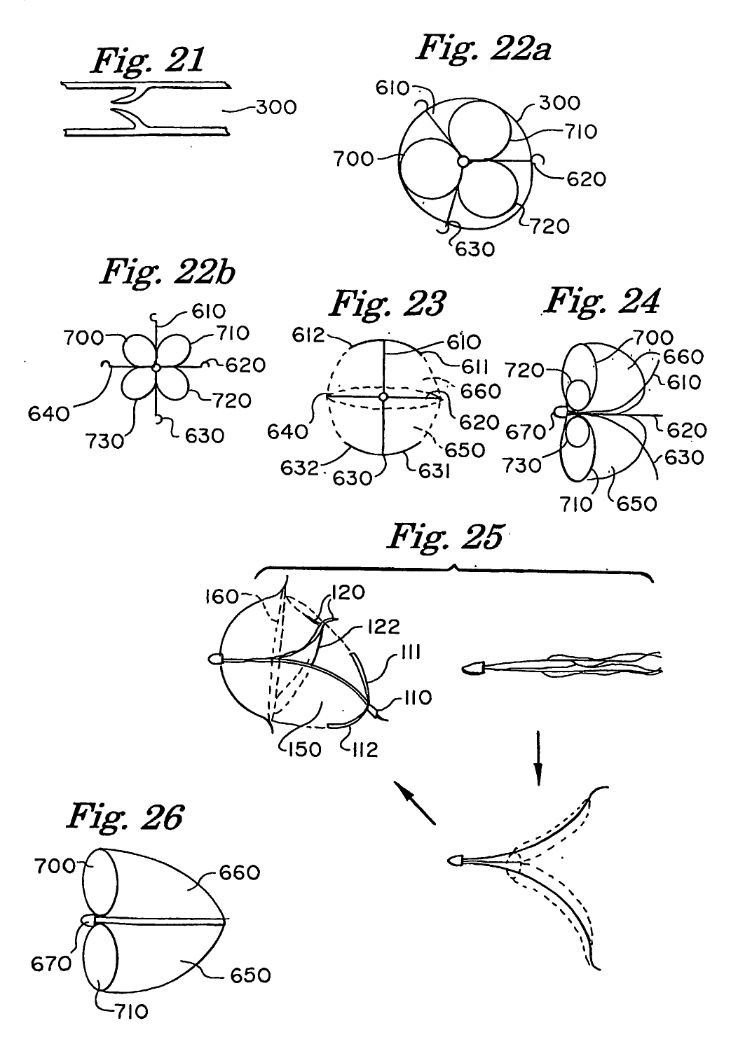

FIG. 21 again shows a cross-section of an exemplary human venous valve 300.

FIG. 22A shows another embodiment of the venous valve with the inner boarder of the vein 300 in relation to the first strut 610 the second strut 620 and the third strut 630. The struts are at approximately equal angles toward one another and shown in this drawing is a first support wing 700 and a second support wing 710 and a third support wing 720. In FIG. 22B shows another embodiment of this design looking from the top in which there is the first strut 610, second strut 620, third strut 630, and fourth strut 640 at approximately equal angles to one another. Also shown in this diagram is the possibility of first support wing 700 and a second support wing 710 and a third support wing 720 and a fourth support wing 730.

FIG. 23 shows the topside view of the FIG. 22b with the first strut 610 having the secondary struts 611 and 612, a second strut 630 with secondary struts 631 and 632, and showing relation to third strut 620 and fourth strut 640 along with the dotted outline of attached membranes 650 and 660.

FIG. 24 is a side view of FIG. 22Bb which shows first strut 610, second strut 620, and third strut 630 in relation to first support wing 700, second support wing 710, third support wing 720, and fourth support wing 730, all of which are connected to the central connector 670. Shown in dotted outline is the relation of the first membrane 650 with its opposite membrane 660.

FIG. 25 starts with another embodiment of FIGS. 7, 8 and 9 first shown in the folded position; next in a partial opening with the first and second strut fully extended; and then ending with the fully extended venous valve. This shows the first strut 110 with secondary struts 111 and 112, the opposite second strut 120 with the first secondary strut 121 and the second secondary strut 122 along with the dotted outline of the membrane 140 and the top edge of the second membrane 160.

FIG. 26 shows a schematic of the first support wing 700 with an attached membrane 660 and a second support wing 710 with an attached membrane 650. The support wings being attached to a connector 670.

What is claimed is:

1. A venous valve replacement for use in a human vein comprising:
a first strut,
a second strut opposite the first strut,
a third strut positioned in an approximately perpendicular plane to the first and second struts,
a fourth strut opposite the third strut,
a central connector for at least one pair of opposite struts, and
at least one membrane forming a valve connecting the distal ends of said struts relative to the central connector.

2. The valve of claim 1 where the overall length of the first and second struts are longer than the overall length of the third and fourth struts.

3. The valve of claim 1 where the first and second struts are formed from a single bio-compatible strand.

4. The valve of claim 1 where the third and fourth struts are formed from a single bio-compatible strand.

5. The valve of claim 1 where the central connector holds all four struts.

6. The valve of claim 5 where the central connector has an aperture which allows sliding the valve apparatus over a guiding device.

7. The valve of claim 1 where at least two membranes are attached to the said distal end of struts.

8. The valve of claim 1 or claim 7 where the membrane is composed of at least one material selected from sclera, biocompatible polymer, and mammalian tissue.

9. A venous valve replacement for use in a human vein comprising:
a first strut,
a second strut opposite the first strut where the first and second struts starting proximal to a central connector curves inward towards the central axis,
a third strut positioned in an approximately perpendicular plane to the first and second strut,
a fourth strut opposite the third strut,
a central connector for at least one pair of opposite struts, and
at least one membrane connecting the distal ends of said struts relative to a central connector.

10. The valve of claim 9 where the overall length of the first and second struts is longer than the overall length of the third and fourth struts.

11. The valve of claim 9 where the first and second struts are formed from a single bio-compatible strand.

12. The valve of claim 9 where the third and fourth struts are formed from a single bio-compatible strand.

13. The valve of claim 9 where the central connector holds all four struts.

14. The valve of claim 9 where the central connector has an aperture which allows sliding the valve over a guiding apparatus.

15. The valve of claim 9 having at least two membranes attached to said distal ends of struts.

16. The valve of claim 9 or claim 15 where the membrane is composed at least of one material selected from sclera, biocompatible polymer, and mammalian tissue.

17. A venous valve replacement for use in a human vein comprising:
a first strut,
a second strut opposite the first strut where the first and second struts, starting proximal to a central connector, curve outward from the central axis,
a third strut laying in approximately perpendicular plane to the first and second struts,
a fourth strut opposite the third strut,
a central connector for at least one pair of opposite struts, and
at least one membrane forming a valve connecting the distal ends of said struts relative to the central connector.

18. The valve of claim 17 where the overall length of the first and second struts is longer than the overall length of the third and fourth struts.

19. The valve of claim 17 where the first and second struts are formed from a single bio-compatible strand.

20. The valve of claim 17 where the third and fourth struts are formed from a single bio-compatible strand.

21. The valve of claim 17 where the central connector holds all four struts.

22. The valve of claim 21 where the central connector has an aperture which allows sliding the valve apparatus over a guiding device.

23. The valve of claim 17 where at least two membranes are attached to the said distal end of struts.

24. The valve of claim 17 or claim 23 where the membrane is composed of at least one or more material selected from sclera, biocompatible polymer and mammalian tissue.

25. A venous valve replacement for use in a human vein comprising:
a first strut,
a second strut opposite the first strut, where said struts each have a pair of secondary struts forming opposites of each other from the distal end of each first and second strut,
a third strut laying in an approximately perpendicular plane to the first and second struts,
a fourth strut opposite the third strut,
a central connector for at least one pair of opposite struts, and
at least one membrane forming a valve connecting the distal ends of said struts relative to the central connector.

26. The valve of claim 25 where the overall length of the first and second struts is longer than the overall length of the third and fourth strut.

27. The valve of claim 25 where the first and second struts are formed from a single biocompatible strand.

28. The valve of claim 25 where the overall length of the third and fourth struts is formed from a single biocompatible strand.

29. The valve of claim 25 where the central connector holds all four struts.

30. The valve of claim 29 where the central connector has an aperture which allows sliding the valve over a guiding apparatus.

31. The valve of claim 25 having are at least two separate membranes attached to said distal ends of struts.

32. The valve of claim 25 or claim 31 where the membrane is composed of at least one or more material selected from sclera, biocompatible polymer, and mammalian tissue.

33. A venous valve replacement for use in a human vein comprising:
at least three struts of equal length approximately equal angles from each other,
at least two support wings,
a central connector for the struts and the support wings, and
at least two membranes forming a valve connected to the struts.
